(19) United States
(12) Patent Application Publication
(10) Pub. No.: US 2026/0199650 A1
(43) Pub. Date: Jul. 16, 2026

(54) INGESTIBLE DEVICE WITH SELF-ORIENTATION CAPABILITY IN AN IN VIVO ENVIRONMENT

(71) Applicant: Verily Life Sciences LLC, Dallas, TX (US)

(72) Inventors: Kye LEE, South San Francisco, CA (US); Joseph DENNIS, Millbrae, CA (US)

(21) Appl. No.: 19/138,048

(22) PCT Filed: Dec. 12, 2023
(86) PCT No.: PCT/US2023/083546

§ 371 Date: Jun. 11, 2025

Related U.S. Application Data
(60) Provisional application No. 63/387,273, filed on Dec. 13, 2022.

(51) Int. Cl. A61M 31/00

(52) U.S. Cl. CPC A61M 31/002; A61M 2205/0238; A61M 2205/106; A61M 2207/00

(57) ABSTRACT

Introduced here is an ingestible device (that is able to deliver medication directly into tissue along the gastrointestinal tract. Administration of a medication stored in the ingestible device may require that the ingestible device orient itself against the tissue. To accomplish this, the ingestible device may have an asymmetrically weighted formfactor. For example, the ingestible device may be weighted along an end (also called the “weighted end”) through which the needle extends in order to promote self-orientation.

### CROSS-REFERENCE TO RELATED APPLICATIONS

This application claims priority to U.S. Provisional Application No. 63/387,273, titled “Ingestible Device with Self-Orientation Capability in an In Vivo Environment” and filed on Dec. 13, 2022, which is incorporated by reference herein in its entirety.

### TECHNICAL FIELD

Various embodiments concern devices designed to administer medications while positioned in a predetermined orientation inside of a living body.

### BACKGROUND

Pharmaceutical drugs (also called “medications”) are an important part of medicine that rely on the continual advancement of pharmacology to diagnose, cure, treat, or prevent disease. The term “administration” is commonly used to refer to the process by which an individual takes a medication. Medications are commonly designed for enteral administration—where the active ingredients enter the body via the gastrointestinal tract—as little oversight is needed. For example, many of the most common medications are intended to be orally administered, with dosages in tablet form, capsule form, or liquid form.

Medications having solid unit dosage forms—namely, tablets and capsules—have several benefits. Not only can medications be designed and/or manufactured to be easier to swallow but also to control the release rate of the active ingredients. Although oral consumption of medications in solid unit dosage form is a fairly straightforward route of administration, absorption of the active ingredients is a complex process.

Most medications that are orally administered are thought to be absorbed in the gastrointestinal tract via passive diffusion or active transport. Passive diffusion is widely considered the more important mechanism, and it depends on transfer of the active ingredients across the mucosa to the circulatory system down a concentration gradient. While transfer itself is largely dependent on the size of the concentration gradient, the rate at which transfer occurs can vary tremendously. For example, the transfer rate may depend on the molecular weight and size of the active ingredients, lipid solubility, mucosal blood flow, mucosal surface area, and mucosal permeability, among other variables. For this reason, it can be difficult to predict how quickly an orally administered medication will be absorbed by a living body.

### BRIEF DESCRIPTION OF THE DRAWINGS

FIG. 1 includes a cross-sectional view of an example of an ingestible device that is designed to administer medication as it travels through a living body, such as a human body or animal body.

FIG. 2A illustrates the ingestible device prior to ingestion by a living body in its “storage state.”

FIG. 2B illustrates the ingestible device in its “insertion state.”

FIG. 2C illustrates the ingestible device in its “injection state.”

FIG. 2D illustrates the ingestible device in its “passable state” following dissolution of the needle.

FIGS. 3A-D include simplified illustrations of the storage state, insertion state, injection state, and passable state, respectively.

FIG. 4 illustrates several possible implementations of a trigger mechanism.

FIG. 5 illustrates how, following ingestion through the mouth, an ingestible device can traverse the esophagus until settling within the stomach.

FIG. 6A includes a simplified illustration of the center of buoyancy and center of mass of an ingestible device with a target vertical orientation.

FIG. 6B includes a simplified illustration of the center of buoyancy and center of mass of an ingestible device with a target horizontal orientation.

FIG. 7 includes an illustration of an ingestible device that includes a capsule to which a ballast is connected.

FIG. 8 illustrates how several designs of an asymmetrically weighted ingestible device reacted in response to agitation of fluid.

FIG. 9 includes a flow diagram of a process for manufacturing a hermetically sealed, asymmetrically weighted structure that is designed for ingestion by a living body.

Various embodiments are shown in the drawings for the purpose of illustration. However, those skilled in the art will recognize that alternative embodiments may be employed without departing from the principles of the present disclosure. Accordingly, while certain embodiments are shown in shown in the drawings, the technologies described herein are amenable to various modifications.

### DETAILED DESCRIPTION

Contemporary research has begun exploring how to improve absorption of orally administered medications. For example, several entities have developed digital pills (also called “smart pills”) that are able to electromechanically effect administration of medications following ingestion. Digital pills commonly monitor aspects of the living body and then use this information to influence operations. The nature of this information can vary, however. For example, a digital pill could include a sensor that aids in the determination of location, and medication may not be administered until the output produced by the sensor indicates that the digital pill is located in a predetermined location.

Despite showing promise, digital pills are still susceptible to error. For example, erroneous output by the sensor could result in the medication being administered in an improper location. As another example, the electromechanical means used to effect administration may malfunction. Further, even if medication is administered in the proper location without issue, absorption still depends on passive diffusion—which, as noted above, can be difficult to predict with accuracy.

There are also some medications that are simply not suitable for oral administration. These medications may cause gastric irritation or experience inconsistent degradation, or these medications may simply be poorly absorbed within the gastrointestinal tract. Historically, many of these medications have been administered via injection. Such an approach has downsides, however, including the risk of infection from piercing the skin, the requirement for a sterile environment, and the like.

Introduced here is an ingestible device (also called a “pill”) that is able to address the aforementioned issues by delivering medication directly into tissue along the gastrointestinal tract. As further discussed below, administration of a medication stored in the ingestible device can be achieved by a combination of features.

First, the ingestible device can include an actuation mechanism that includes (i) a plunging mechanism to which a needle is connected and (ii) a dissolvable trigger mechanism that holds the plunging mechanism in a first position. The term “trigger mechanism” may be used to refer to a mechanical component that physically inhibits movement of the plunging mechanism (and therefore, administration of medication via the needle). Following ingestion, the trigger mechanism may be exposed to body fluids to provoke dissolution. When the trigger mechanism dissolves, the plunging mechanism may move from the first position to a second position, thereby causing the needle to extend into tissue against which the ingestible device is lodged. Such a feature allows the ingestible device to actuate at a predictable time—measured with respect to ingestion—without the need for external stimulus (e.g., a signal originating outside of the body) or internal stimulus (e.g., an output produced by a sensor).

Second, the needle may also be dissolved through exposure to body fluids. In operation, the bevel end of the needle may extend into tissue following dissolution of the trigger mechanism of the actuation mechanism. Over an interval of time, the medication in the ingestible device can be delivered into the tissue via the needle. The needle may be designed such that dissolution occurs after a predetermined amount of time has elapsed. The bevel end is sharp enough to pierce the tissue, and therefore, must be accommodated as the ingestible device traverses the gastrointestinal tract. Having the needle dissolve eliminates the need for the needle to be retracted back into the ingestible device in order to ensure safe passage through the gastrointestinal tract.

Any ingestible device is designed to administer medication directly into tissue along the gastrointestinal tract must orient itself against the tissue. For example, if the medication is to be administered into the stomach lining, the ingestible device must be in intimate contact with the stomach lining such that its needle is aligned with the surface in a roughly orthogonal manner. Establishing and then maintaining this alignment is not a trivial task, however. Digital pills have traditionally relied on passive locomotion, allowing the digestive system to do all of the work in moving a digital pill through a living body. Passive locomotion can be unpredictable, however, as the orientation within a given organ is not only arbitrary but can also change over time. For example, the orientation of a digital pill that is located in the stomach can change as the digital pill moves about the stomach. Some digital pills have begun utilizing more active locomotion in an attempt to address the drawbacks of passive locomotion. For example, a digital pill could include a propellor that, in operation, causes propulsion of the digital pill, or a digital pull could include a magnet that can be activated by a magnetic field located external to the living body. Various approaches to actively locomoting digital pills have been proposed. All of these approaches require the addition of components that are costly, susceptible to malfunction, or increase risk to the living body.

Accordingly, the ingestible device introduced herein may have an asymmetrically weighted formfactor. As further discussed below, the ingestible device may be weighted along an end (also called the “weighted end”) through which the needle extends in order to address the self-orientation problem. To further stabilize the ingestible device, the weighted end may include a mucoadhesive coating that improves, at least temporarily, adherence to the surface of tissue.

### Terminology

References in the present disclosure to “an embodiment” or “some embodiments” means that the feature, function, structure, or characteristic being described is included in at least one embodiment. Occurrences of such phrases do not necessarily refer to the same embodiment, nor do they necessarily refer to alternative embodiments that are mutually exclusive of one another.

The term “based on” is to be construed in an inclusive sense rather than an exclusive sense. That is, in the sense of “including but not limited to.” Thus, the term “based on” is intended to mean “based at least in part on” unless otherwise noted.

The terms “connected,” “coupled,” and variants thereof are intended to include any connection or coupling between two or more elements, either direct or indirect. The connection or coupling can be physical, logical, or a combination thereof. For example, elements may be electrically or communicatively connected to one another despite not sharing a physical connection.

The term “module” may refer broadly to software, firmware, hardware, or combinations thereof. Modules are typically functional components that generate one or more outputs based on one or more inputs. A computer program may include or utilize one or more modules. For example, a computer program may utilize multiple modules that are responsible for completing different tasks, or a computer program may utilize a single module that is responsible for completing multiple tasks.

When used in reference to a list of items, the word “or” is intended to cover all of the following interpretations: any of the items in the list, all of the items in the list, and any combination of items in the list.

The term “biocompatible” means not harmful to living tissue. Accordingly, the term “biocompatible material” may be used to refer to any material that is not harmful to living tissue, whether its biocompatibility is presently known or not known.

The term “compressed state” may be used to refer to any state in which a spring is at least partially compressed. Meanwhile, the term “uncompressed state” may be used to refer to any state in which the spring is substantially uncompressed. Those skilled in the art will recognize that whether a spring is fully compressed or partially compressed while constrained- or fully uncompressed or substantially uncompressed while not constrained—may depend on the nature (e.g., design and size) of the spring.

### Overview of Ingestible Device

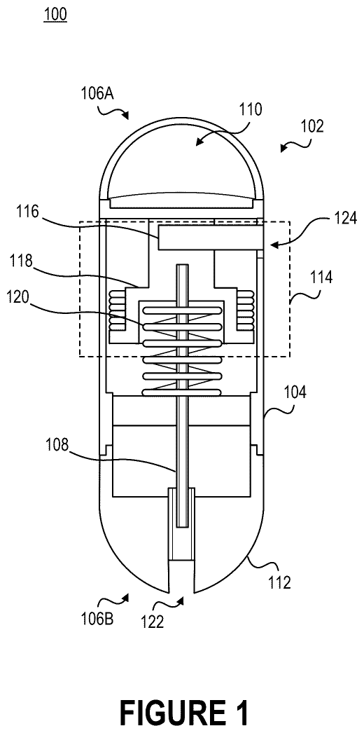

FIG. 1 includes a cross-sectional view of an example of an ingestible device 100 that is designed to administer medication as it travels through a living body, such as a human body or animal body. Note that FIG. 1 and other illustrations in the present disclosure are not drawn to scale. Features may be shown significantly enlarged for greater clarity.

As shown in FIG. 1, the ingestible device 100 can include a capsule 102 with a cylindrical body 104 and atraumatically shaped ends 106A-B. One example of an atraumatically shaped end is a rounded shape that does not cause damage upon contacting living tissue, such as the roughly hemispherical ends shown in FIG. 1. This geometric shape is commonly called a ‘spherocylinder.” While the ingestible device 100 shown in FIG. 1 has roughly hemispherical ends, the ingestible device 100 could have other atraumatically shaped ends in other embodiments. For example, at least one end of the capsule 102 may have a flat portion that can lie against the living tissue. The cylindrical body 104 and atraumatically shaped ends 106A-B may be referred to as the “structural components” of the capsule 102. To avoid contamination of an internal cavity defined by the cylindrical body 104 and/or the atraumatically shaped ends 106A-B, the structural components may be hermetically connected to one another.

Note that in some embodiments, the cylindrical body 104 is integrally formed with one of the atraumatically shaped ends. For example, the cylindrical body 104 may be integrally formed with atraumatically shaped end 106A, and therefore these structural components may not need to be connected to one another. Instead, components could be installed within these structural components and then atraumatically shaped end 106B may be connected thereto.

In some embodiments, these structural components comprise the same material. For example, these structural components may comprise plastic, metal, metal alloy, ceramic, polymer, or another biocompatible material, such as naturally derived materials that have comparable mechanical properties to plastics. In other embodiments, these structural components comprise different materials. For example, the atraumatically shaped end 106B through which the needle 108 extends may be comprised of a polymer or metal alloy, while the other atraumatically shaped end 106A and cylindrical body 104 may be comprised of plastic. A large disparity in weight may be helpful in ensuring that the ingestible device 100 is properly aligned with respect to the living tissue into which medication is to be injected, as further discussed below. Moreover, these structural components may have a coating that inhibits exposure to, and degradation from, body fluids. For example, these structural components may be coated with a polymer, a sugar, or a sugar alcohol, via a dip process or spray process, and the coating may improve safety, durability, or operational efficiency of the ingestible device 100. For example, a polymer coating can provide lubrication to aid in passage through the esophagus but may be chemically designed to denature, dissolve, or otherwise degrade in the stomach, yet still remain robust while in the mouth and esophagus and during normal handling.

Regardless of whether these structural components comprise the same material or different materials, the capsule 102 can be formed in various ways. For example, these structural components could be machined, injection molded, printed (e.g., with a three-dimensional printer), or otherwise formed to accommodate components of the ingestible device 100.

As shown in FIG. 1, atraumatically shaped end 106A may be largely or entirely empty to provide buoyancy. Here, for example, the inner surface of atraumatically shaped end 106A defines a cavity 110 that is vacant. However, relatively lightweight components could be situated within atraumatically shaped end 106A without meaningfully affecting the buoyancy. Such a design may cause the ingestible device 100 to naturally be oriented longitudinally when in body fluids, as further discussed below.

Meanwhile, a ballast 112 may be situated within atraumatically shaped end 106B. The ballast 112 may comprise any material that is able to provide stability. Examples of such materials include metals, metal alloys (e.g., stainless steel, titanium alloys, cobalt-chromium alloys), ceramics (e.g., tungsten carbide), and the like. In FIG. 1, the ballast 112 is connected to the longitudinal segment of the capsule 102 in such a manner that the ballast forms atraumatically shaped end 106B. In such embodiments, the ballast 112 is partially or entirely exposed to body fluids following ingestion of the ingestible device 100, and therefore may comprise a biocompatible material. However, in some embodiments, the ballast 112 is positioned inside atraumatically shaped end 106B. Because the ballast 112 is not exposed to body fluids in such embodiments, the ballast 112 may or may not comprise a biocompatible material.

As further discussed below, at least one of these structural components could comprise a dissolvable material. Assume, for example, that the capsule 102 includes atraumatically shaped end 106A and cylindrical body 104, but no atraumatically shaped end 106B (and therefore, the ballast 112 is exposed to body fluids). Atraumatically shaped end 106A and/or cylindrical body 104 may comprise a material that dissolves following exposure to body fluids for a predetermined amount of time. The material may be a water-soluble polymer or a mixture or combination of soluble and insoluble materials. Examples of soluble materials include polyethylene glycol (PEG), polyvinylpyrrolidone (PVP), Dextran, polyethylene oxide, polyvinyl alcohol (PVA), polyacrylic acid (PAA), hydroxypropyl methylcellulose (HPMC), etc. Examples of insoluble materials include polycarbonate, polystyrene, etc. Generally, the predetermined amount of time is sufficiently long that (i) a trigger mechanism 116 of an actuation mechanism 114 and (ii) the needle 108 dissolve before the predetermined amount of time elapses. Dissolution of atraumatically shaped end 106A, atraumatically shaped end 106B, or cylindrical body 104 may be helpful in decreasing the size of the ingestible device 100, making it easier for the ingestible device 100 to pass through the gastrointestinal tract after medication has been administered.

As shown in FIG. 1, atraumatically shaped end 106B can include an aperture 122 through which the needle 108 is able to extend. An actuation mechanism 114 may be responsible for moving the needle 108 from a first position in which its bevel end is located inside the capsule 102 to a second position in which its bevel end is located outside the capsule 102. As further discussed below, the actuation mechanism 114 may include a trigger mechanism 116 that, following ingestion, is exposed to body fluids through another aperture 124 in the capsule 102, a plunger mechanism 118 that is held in a first position by the trigger mechanism 116, and a spring 120 that is held in a compressed state by the plunger mechanism 118 while in the first position. The trigger mechanism 116 may comprise a material that dissolves following exposure to body fluids. Following dissolution of the trigger mechanism 116, the plunging mechanism 118 may move from the first position to a second position due to the spring 120 transitioning from the compressed state to an uncompressed state. The needle 108 may be connected—either directly or indirectly—to the plunger mechanism 118, and therefore movement of the plunger mechanism 118 may correspond to movement of the needle 108. The term “plunger mechanism” may be used to refer to a mechanical component that causes medication to be administer via rapid repositioning. Approaches to moving the needle 108 are discussed in greater detail below.

The capsule 102 may have any of a variety of different sizes, such as any of those sizes listed in Table I. Generally, the size of the capsule 102 depends on its contents (e.g., the amount of medication to be stored therein).

TABLE I
Example sizes of capsules.

| Size | 000 | 00 | 0 | 1 | 2 | 3 | 4 |
|---|---|---|---|---|---|---|---|
| Internal Cavity | 1.37 | 0.91 | 0.68 | 0.50 | 0.37 | 0.30 | 0.21 |
| Capacity (ml) | | | | | | | |
| Length (mm) | 35 | 30 | 29 | 26.5 | 24 | 21.5 | 19.5 |
| Diameter (mm) | 15 | 13.5 | 11 | 7 | 6.5 | 6 | 5.5 |

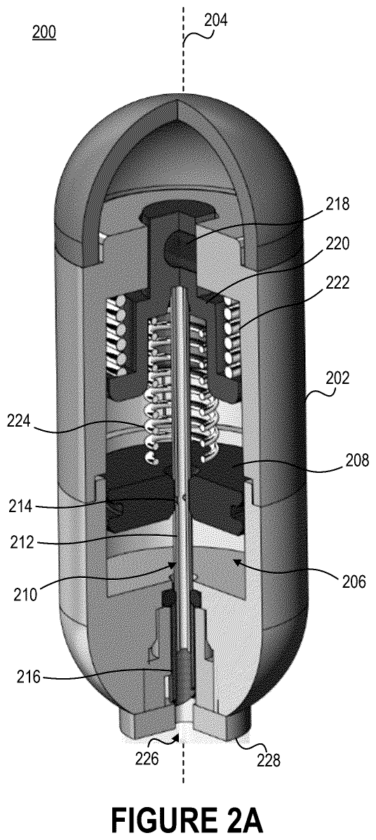

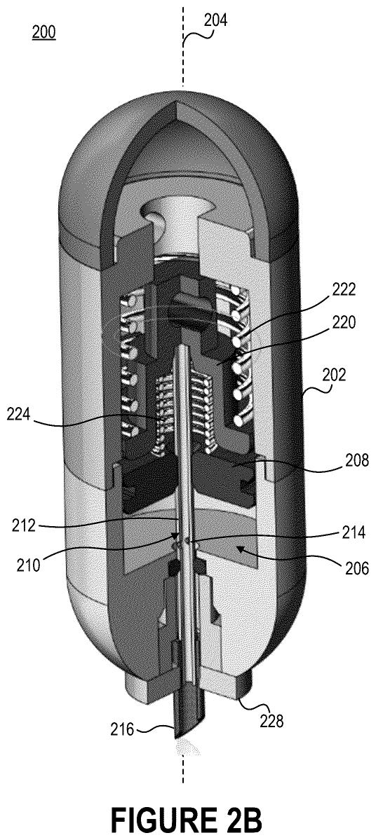

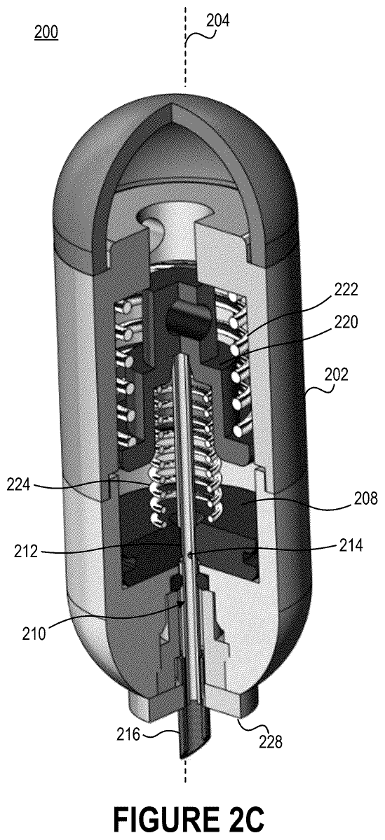

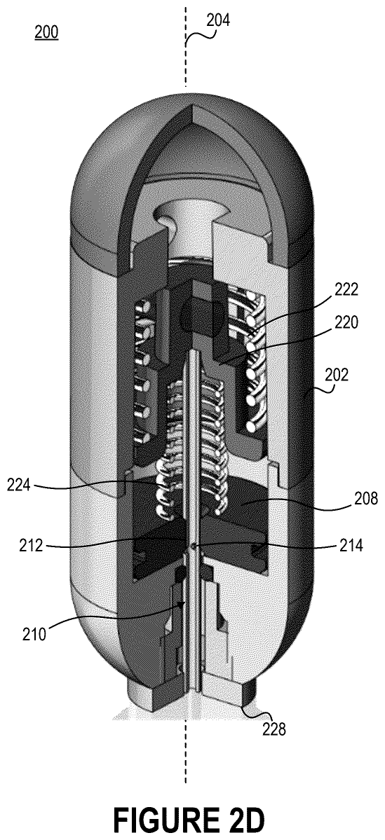

FIGS. 2A-D illustrate an ingestible device 200 at different stages of administration.

FIG. 2A illustrates the ingestible device 200 prior to ingestion by a living body. At this stage, the ingestible device 200 may be described as being in the “storage state.” As discussed above, the ingestible device 200 may comprise a capsule 202 having a central longitudinal axis 204 (or simply “central axis”) defined therethrough. The capsule 202 may have a substantially cylindrical segment that is interconnected between a pair of rounded ends. In embodiments where the rounded ends are in the form of hemispheres, this shape may be called a “spherocylinder.” Inside the substantially cylindrical segment, a reservoir 206 may store medication—generally in liquid form. The volume of medication that is storable in the reservoir 206 may depend on the size of the capsule 202. Generally, the reservoir 206 is able to store between 25-200 microliters (μL) (and preferably 50-100 μL). As shown in FIG. 2A, the reservoir 206 may be partially defined by a seal 208. The bottom surface of the seal 208—which may define a periphery of the reservoir 206—may be roughly orthogonal to the central axis 204 while in a first position (also called an “upper position”).

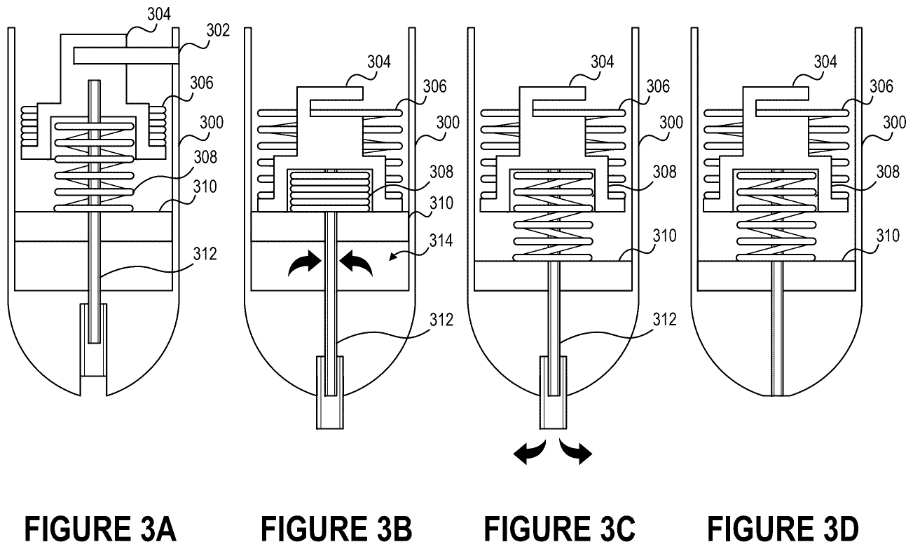

A needle 210 may be arranged roughly along the central axis 204. The needle 210 may include a hollow shaft 212 with a port 214 defined therein and a bevel end 216. Medication in the reservoir 206 may be able to enter the hollow shaft 212 through the port 214 and exit through the bevel end 216. In FIG. 3A, the needle 210 is located in a first position corresponding to a storage state. When the needle 210 is located in the first position, the port 214 may be obscured so as to prevent medication from entering the hollow shaft 212. For example, the port 214 may be covered by the seal 208 through which the needle 210 extends, as shown in FIG. 2A.

In FIGS. 2B-C, the bevel end 216 has a 45° regular bevel. However, the bevel end 216 could be designed otherwise depending on the tissue into which the needle 210 is to be inserted, the force with which the needle 210 will be inserted into the tissue, etc. Accordingly, the bevel end 216 could have a 22° bevel, 30° bevel, 35° bevel, 45° bevel, etc. In some embodiments, the end 216 may not be beveled at all, instead having a 90° blunt end. Moreover, the bevel end 216 could have a long bevel, medium bevel, short bevel, multi-bevel, or scalpel bevel. Further, the needle 210 could have other forms in some embodiments. For example, the ingestible device 200 could have multiple “microneedles” of relatively small size that are arranged in the form of an array, and the entire microneedle array could be actuated in accordance with the approach described herein. As another example, the ingestible device 200 could include a single needle, such as a macroneedle (e.g., comprised of epoxy), that has one or more apertures (also called “gills”) along its length through which medication can exit. As another example, the ingestible device could include a barbed projection having one or more apertures through which medication can exit. These apertures may be arranged along its length or near its end (e.g., proximate to the barbs that embed into tissue to inhibit extraction). In such embodiments, the barbed projection may be dissolvable to ensure that the ingestible device 200 is able to readily detach from the tissue into which the medication is administered.

As noted above, the ingestible device 200 may include an actuation mechanism that, in operation, causes the needle 210 to move along the central axis 204 from the first position in which the bevel end 216 is located inside the capsule 202 to a second position in which the bevel end 216 is located outside the capsule 202. The actuation mechanism can include (i) a trigger mechanism 218, (ii) a plunger mechanism 220, and (iii) a first spring 222. In embodiments where the ballast forms one of the atraumatically shaped ends, the ballast may have an aperture defined therethrough along the central axis 204, so as to accommodate actuation of the needle 210 along the central axis 204.

Following ingestion, the trigger mechanism 218 may be exposed to body fluids through an aperture (not shown) in the capsule 202. The trigger mechanism 218 can comprise a material that dissolves following exposure to the body fluids for a predetermined amount of time. The material could be a dissolvable polymer, for example. The trigger mechanism 218 could have various forms depending on the design of the actuation mechanism (and more specifically, the plunger mechanism 220 that is held in place by the trigger mechanism 218). In FIG. 2A, for example, the trigger mechanism 218 is a pin that extends laterally through an opening (e.g., a hole or slot) in the plunger mechanism 220. The aperture in the capsule 202 through which body fluids are able to contact the trigger mechanism 218 could be located in nearly any position along the periphery of the capsule 202. For example, the aperture may be along the axial axis of the pin, such that the aperture is proximate to one end of the pin. As another example, the aperture may be along a radial axis of the pin, such that the aperture is centrally located along the length of the pin. More than one aperture may be defined through the capsule 202 if the trigger mechanism 218 is to be exposed to greater amounts of body fluid (e.g., to quicken dissolution). For example, the aperture may take the form of a slot that extends radially around at least part of the periphery of the capsule 202.

Further, the size of the trigger mechanism 218 may be altered to achieve dissolution at a desired rate. Generally, thinner trigger mechanisms will dissolve more quickly than thicker trigger mechanisms. The trigger mechanism 218 may have a diameter between 1.0-2.5 millimeters. The length of the trigger mechanism 218 may depend on the size of the capsule 202. For example, the trigger mechanism 218 may have a length between 3.0-10.0 millimeters.

As shown in FIG. 2A, the plunger mechanism 220 may initially be held in a first position by the trigger mechanism 218. While in the first position, the plunger mechanism 220 may hold the first spring 222 (also called the “insertion spring”) in a compressed state. The needle 210 can be connected to the plunger mechanism 220, and therefore, movement of the plunger mechanism 220 may correspond to movement of the needle 210 as further discussed below with reference to FIGS. 2B-C.

Another spring 224 (called the “second spring” or “injection spring”) can be interconnected between the plunger mechanism 220 and seal 208. When the plunger mechanism 220 in is the first position—shown in FIG. 2A—the second spring 224 can be in an uncompressed state or lightly compressed state. When the second spring 224 is in an uncompressed state, the seal 208 will be suspended such that little or no pressure is applied to the medication in the reservoir 206. The tandem nature of the first and second springs 222, 224 may allow the medication to be stored in a low-pressure configuration. It also allows the insertion of the needle 210 into the tissue to be decoupled in time and force from the delivery of the medication into the tissue, allowing for more robust insertion and more reliable timing of medication delivery.

In some embodiments, a mucoadhesive disk 228 is secured along the end of the capsule 202 weighted by the ballast, as shown in FIG. 2A. The term “mucoadhesion” is commonly used to refer to the adhesion that occurs between two surfaces, one of which is mucosal in nature. The mucoadhesive disk 228 may comprise any material that is able to improve, at least temporarily, adherence of the ingestible device 200 to the tissue into which medication is to be ejected. The mucoadhesive disk 228 may comprise a polymer, for example, that has hydrophilic groups (e.g., hydroxyl, carboxyl, amide, or sulfate) that attach to the tissue via interactions such as hydrogen bonding, hydrophobic interactions, or electrostatic interactions. For example, the polymer may be coated along the mucoadhesive disk 228, or the mucoadhesive disk 228 may be comprised entirely of the polymer. In some embodiments, the polymer is dissolvable. Dissolution of the polymer may allow the mucoadhesive disk 228 to more readily detach from the tissue. For example, adhesiveness could simply lessen due to dissolution of the polymer, or dissolution of the polymer could result in dissolution of the mucoadhesive disk 228 itself.

Referring now to FIG. 2B, following dissolution of the trigger mechanism 218, the plunging mechanism 220 can move from the first position to a second position in response to the first spring 222 transitioning from the compressed state to an uncompressed state. In FIG. 2B, dissolution of the trigger mechanism 218 causes the plunging mechanism 220 to move “downward” along the central axis 204. Because the needle 210 is connected to the plunging mechanism 220, movement of the plunging mechanism 220 can correspond to movement of the needle 210 along the central axis 204. Specifically, the bevel end 216 of the needle 210 can extend through another aperture 226 in the capsule 202. In the event that the ingestible device 200 is located adjacent to tissue, such movement of the needle 210 may result in the bevel end 216 piercing the surface of the tissue. Accordingly, FIG. 2B illustrates the ingestible device 200 in its “insertion state.”

In FIG. 2B, the needle 210 is located in a second position corresponding to an insertion state. When the needle 210 is located in the second position, the port 214 may be accessible such that medication can enter the hollow shaft 212. At a high level, the port 214 is located within the periphery of the reservoir 206 while the needle 210 is located in the second position. However, the needle 210 is preferably designed such that the port 214 is located near the “bottom” of the reservoir 206, as shown in FIG. 2B. Medication may be able to more easily flow into the hollow shaft 212 when the port 214 is located nearer the “bottom” of the reservoir 206. Moreover, locating the port 214 near the “bottom” of the reservoir 206 will allow more medication to be administered, as the port 214 is less likely to be obscured, for example, by the seal 208 as it moves “downward” along the central axis 204 from the first position (also called the “upper position”) to a second position (also called a “lower position”). The seal 208 is located in the upper position in FIG. 2A, while the seal 208 is located in the lower position in FIG. 2C.

As shown in FIG. 2B, the second spring 224 can be compressed due to “downward” movement of the plunging mechanism 220 along the central axis 204. This compression is due to the force applied to the bottom surface of the seal 208 by the medication stored in the reservoir 206. At a high level, the medication is sufficiently constrained (and therefore, pressurized) that force is applied against the bottom surface of the seal 208.

Referring now to FIG. 2C, as medication flows into the needle 210 through the port 214 and out of the needle 210 through the bevel end 216, the amount of medication in the reservoir 206 will lessen (and therefore, the force applied against the bottom surface of the seal 208 will also lessen). Accordingly, FIG. 2C illustrates the ingestible device 200 in its “injection state.” Over time, the second spring 224 will transition from the compressed state to an uncompressed state. Such movement will “push” the medication through the port 214 into the needle 210. Generally, the second spring 224 is designed such that when the second spring 224 returns to the uncompressed state, the seal 208 is located near the bottom of the reservoir 206, as shown in FIG. 2C. Said another way, the second spring 224 may be designed—in combination with the seal 208, first spring 222 and plunger mechanism 220—so that most, if not all, of the medication is administered into tissue through the bevel end 216 of the needle.

Note that, in operation, the insertion stage and injection stage shown in FIG. 2B and FIG. 2C, respectively, tend to occur in rapid succession. For example, the ingestible device 200 may only be in the insertion stage momentarily (e.g., several dozen or hundred milliseconds), as the second spring 224 rapidly compresses as shown in FIG. 2B and then expands as shown in FIG. 2C.

As mentioned above, the needle 210 may comprise a material that dissolves following exposure to body fluids. In operation, the bevel end 216 of the needle 210 may extend into tissue during the insertion stage, and medication may flow through the bevel end 216 of the needle 210 into the tissue during the injection stage. The ingestible device 208 (and more specifically, its actuation mechanism, reservoir 206, seal 208, and needle 210) can be designed so that the medication is mostly, if not entirely, administered over an interval of time having a known length. For example, the interval of time may be several seconds to several minutes. As further discussed below, the needle 210 can be designed such that dissolution occurs after the interval of time has elapsed. Because the bevel end 216 is sharp enough to pierce the tissue, it must be accommodated if the ingestible device 200 will exit the living body by traversing the gastrointestinal tract. Having the needle 210 dissolve eliminates the need for the needle 210 to be retracted back into the capsule 202 in order to ensure safe passage through the gastrointestinal tract. FIG. 2D illustrates the ingestible device 200 in its “passable state” following dissolution of the needle 210.

FIGS. 3A-D include simplified illustrations of the storage state, insertion state, injection state, and passable state, respectively. For simplicity, only the actuation mechanism—including the trigger mechanism 302, plunger mechanism 304, and first spring 306—second spring 308, seal 310, and needle 312 are shown with respect to the capsule 300.

Following ingestion, the trigger mechanism 302 can be exposed to body fluids through an aperture in the capsule 300. The trigger mechanism 302 can hold the plunger mechanism 304 in a first position, and while the plunger mechanism 304 is in the first position, the plunger mechanism 304 may hold the first spring 306 in a compressed state, as shown in FIG. 3A. Further, because the needle 312 is connected to the plunger mechanism 304, the trigger mechanism 302 can also hold the needle 312 in a first position-albeit indirectly via the plunger mechanism 304. While the needle 312 is in the first position, (i) the port through which medication enters can be obscured and (ii) the bevel end through which the medication exits can be fully retained within the capsule 300. For example, the port may be centrally located along the length of the needle 312, such that the port is covered by the seal 310.

As mentioned above, the trigger mechanism 302 can be comprised of a material that dissolves following exposure to body fluids. Following dissolution of the trigger mechanism 302, the plunger mechanism 304 can move from the first position to a second position, as shown in FIG. 3B. Movement of the plunger mechanism 304 may be caused by the first spring 306 transitioning from the compressed state to an uncompressed state. Such action may cause the bevel end of the needle 312 to extend through an aperture in the capsule 300, as shown in FIG. 3B. The stroke length of the needle 312 can depend on various factors, including the length of the needle 312, the length of the first spring 306, the amount of compression of the first spring 306, and the like. However, the length of the stroke during the insertion stage—commonly called the “insertion stroke”—may be between 2-4 millimeters (and preferably 2.5-3.5 millimeters).

As the plunger mechanism 304 moves toward the second position, the second spring 308 may become more compressed. Said another way, movement of the plunger mechanism 304 may cause the second spring 308 to transition from the uncompressed state to a compressed state. Note that the second spring 308 may not necessarily become fully compressed as shown in FIG. 3B, but may instead be partially compressed due to the resistive force applied by the seal 310. Thereafter, the second spring 308 will transition from the compressed state back to the uncompressed state. As shown in FIG. 3C, reversion of the second spring 308 to the uncompressed state can cause the seal 310 to move from a first position (FIG. 3B) to a second position (FIG. 3C). While not shown in detail here, the seal 310 may move through a reservoir 314, “pushing” medication in the reservoir 314 into the needle 312 through a port. The medication can travel through the hollow shaft of the needle 312 and then be ejected through the bevel end of the needle 312.

In some embodiments, movement of the seal 310 does not cause any further movement of the needle 312. Said another way, the stroke length of the needle 312 may not lengthen during the injection stage in some embodiments. However, in other embodiments, the needle 312 is further extended as the seal 310 moves. This may be caused by further movement of the plunger mechanism 304 as the reservoir is emptied of medication. The length of the stroke during the injection stage—commonly called the “injection stroke”—may be between 1-3 millimeters (and preferably 1.5-2.0 millimeters). The injection stroke can be optimized based on the target volume of medication to be delivered and the configuration of the reservoir 314. For example, because a capsule of 000 size has a larger cross-sectional area available for the reservoir 314, less stroke may be required to deliver the same volume of medication in comparison to a capsule of 0 size.

The needle 312 can also be comprised of a material that dissolves, softens, or otherwise denatures following exposure to body fluids to reduce the potential for tissue damage while passing through the gastrointestinal tract. Accordingly, the needle 312—or at least a portion thereof (e.g., the bevel end)—may dissolve. Generally, the needle 312 is designed or constructed such that sufficient time (e.g., 2-30 minutes, and preferably 5-10 minutes) elapses for administration purposes before dissolution begins. As shown in FIG. 3D, dissolution of the needle 312 allows that end of the capsule 300 to become atraumatic once again.

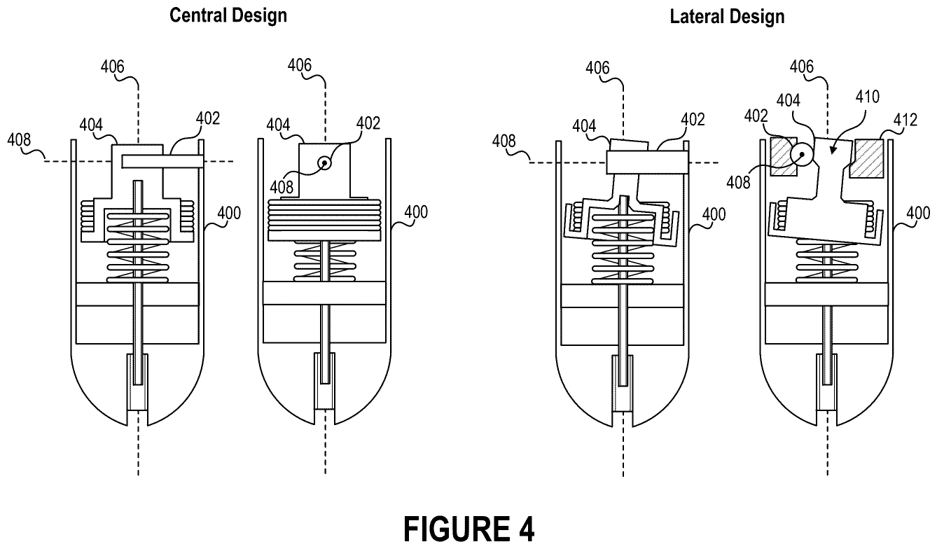

FIG. 4 illustrates several possible implementations of a trigger mechanism 402. Specifically, FIG. 4 illustrates a “central design” where the trigger mechanism 402 extends into the plunger mechanism 404 and a “lateral design” where the trigger mechanism 402 runs alongside the plunger mechanism 404. Those skilled in the art will recognize that these implementations are intended to illustrate rather than limit the nature of the trigger mechanism 402.

In the “central design,” the trigger mechanism 402 can extend into an opening in the plunger mechanism 404. The opening could be a notch as shown in FIG. 4, or the opening could be an aperture that extends entirely through the plunger mechanism 404 roughly along a latitudinal axis 408. When held in place by the trigger mechanism 402, the plunger mechanism 404 may be oriented lengthwise roughly along a longitudinal axis 406. As mentioned above, following ingestion, the trigger mechanism 402 may be exposed to body fluids through an aperture in the capsule 400, and upon dissolving, the plunger mechanism 404 may move “downward” along the longitudinal axis 406 such that the bevel end of the needle extends through the capsule 400.

In the “lateral design,” the trigger mechanism 402 can run alongside the plunger mechanism 404. As shown in FIG. 4, the plunger mechanism 404 may be tilted (e.g., by 5-10 degrees with respect to the longitudinal axis 406), such that a latching component 410 engages a structural component 412. The structural component 412 may be partially complementary to the latching component 410, such that movement of the plunging mechanism 404 is inhibited by engagement between those components while the trigger mechanism 402 is in place. Generally, the latching component 410 and structural component 412 are designed to permit a maximum axial translation along the latitudinal axis 408 between 0.1-0.5 millimeters (and preferably about 0.2-0.3 millimeters). The structural component 412 could be affixed to the inner surface of the capsule 400, or the structural component 412 could be part of the capsule 400. For example, the structural component 412 may be representative of the inner surface of the cylindrical body (e.g., cylindrical body 104 of FIG. 1) or atraumatically shaped end (e.g., atraumatically shaped end 106A of FIG. 1). At a high level, the trigger mechanism 402 may “pin” the latching component 410 of the plunger mechanism 404 against the structural component 412.

As shown in FIG. 4, the trigger mechanism 402 may tip the plunger mechanism 404 off the longitudinal axis 406, and as the trigger mechanism 402 dissolves, the design of the plunger mechanism 404 (and more specifically, the latching component 410) may allow for self-alignment with the longitudinal axis 406 and release from its “pinned” position. Following dissolution of the trigger mechanism 402, diametric reduction of the structural component 412 may permit actuation of the plunger mechanism 404. The diametric reduction may vary based on the design (e.g., form and dimensions) of the latching component 410. For example, if the latching component 410 has a roughly inverted bell form, then diametric reduction of 0.3-0.7 millimeters may permit actuation. Following dissolution of the trigger mechanism 402, the plunger mechanism 404 may move “downward” along the longitudinal axis 406 and laterally along the latitudinal axis 408 toward the longitudinal axis 406. Accordingly, the plunger mechanism 404 may “straighten out” as it moves “downward.”

Having a dissolvable trigger mechanism may be an important feature of the ingestible device, as it allows actuation of the needle to be done “passively” in the sense that active actuation (e.g., with a motor) is not necessary. In the “lateral design,” while the trigger mechanism 402 prevents movement of the plunger mechanism 404 prior to dissolution, the trigger mechanism 402 need not be comprised of materials having high strength. Simply put, when the trigger mechanism 402 is positioned alongside the plunger mechanism 404, little material strength is required because this design eliminates dependence on reduction of shear strength. Further, the trigger mechanism 402 may be axi-symmetric with this design, which may ease the manufacturing and assembling processes.

### Overview of Passive Locomotion and Orientation Processes

### A. Passive Locomotion

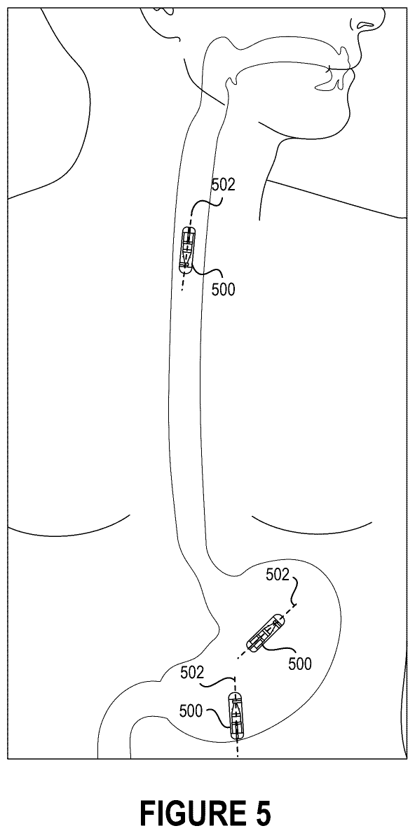

FIG. 5 illustrates how, following ingestion through the mouth, an ingestible device 500 can traverse the esophagus until settling within the stomach. Once inside the stomach, the ingestible device 500 can naturally situate itself in a longitudinal arrangement (also called the “vertical arrangement”). The ingestible device 500 may have a central axis 502 defined therethrough, and when the ingestible device 500 is in the vertical arrangement, the central axis 502 may be roughly orthogonal to the surface 504 of the tissue into which medication is to be injected.

As mentioned above, the ingestible device 500 may naturally situate itself in the vertical arrangement along the bottom of the stomach. Generally, this is accomplished by designing a first end (e.g., atraumatically shaped end 106A of FIG. 1) of the ingestible device 500 to provide buoyancy and a second end (e.g., atraumatically shaped end 106B of FIG. 1) of the ingestible device 500 to provide ballast. For example, the first end may be largely or entirely empty to provide buoyancy, while the second end may include a high-density component (e.g., comprised of metal, metal alloy, ceramic, etc.) that ensures proper orientation within the stomach.

In some embodiments, a mucoadhesive is coated along at least the second end of the ingestible device 500. Said another way, the mucoadhesive may be coated along at least an exterior surface of one end of the ingestible device 500. Like the mucoadhesive disk 228 of FIGS. 2A-D, the mucoadhesive coating can adhere the ingestible device 500 to the tissue to ensure more reliable delivery of the medication contained in the ingestible device 500. The mucoadhesive coating may comprise a polymer, for example, that has hydrophilic groups (e.g., hydroxyl, carboxyl, amide, or sulfate) that attach to the tissue via interactions such as hydrogen bonding, hydrophobic interactions, or electrostatic interactions. Note that in some embodiments, the mucoadhesive coating is constructed or applied such that adhesiveness decreases over time. Assume, for example, that the ingestible device 500 is designed such that its needle dissolves following a first interval of time. It may be desirable for the mucoadhesive coating to lose adhesiveness after—or shortly before—expiration of the first interval of time, so that the ingestible device 500 more readily detaches from the surface 504 of the tissue. Accordingly, the mucoadhesive coating may be designed to dissolve, allowing the ingestible device 500 to complete transit through the gastrointestinal tract intact.

Such an approach to gastric delivery of medication may be preferable to traditional medications with solid unit dosage forms that rely heavily on passive diffusion. For example, esophageal transit time of the ingestible device 500 may be 5 minutes or less following ingestion and medication may be administered shortly thereafter (e.g., within 5-20 minutes of ingestion, and preferably within 5-10 minutes of ingestion), while traditional medications may not be absorbed for 120 minutes or more. Consequently, medications can be administered- and take effect-more quickly using the ingestible device introduced here. There is also improved safety margin by administering medication in the stomach, as the thickness of gastric tissue generally averages about 5 millimeters in comparison to intestinal tissue that generally averages about 1 millimeter.

### B. Passive Orientation

As mentioned above, any ingestible device that is designed to administer medication directly into tissue along the gastrointestinal tract must orient itself-either passively or actively-against the tissue. Consider, for example, a scenario in which medication is to be administered into the stomach lining as shown in FIG. 5. The ingestible device must be in intimate contact with the stomach lining such that its needle is aligned with the surface, preferably in a roughly orthogonal manner.

Establishing and then maintaining this alignment is not a trivial task, however. Relying entirely on passive locomotion has historically been problematic, as the orientation within a given organ is not only arbitrary but can also change over time. To address this issue, several entities have developed propulsion mechanisms that are able to locomote ingestible devices in an active manner. However, these attempts at active locomotion tend to be costly, susceptible to malfunction, or increase risk to the living body.

Accordingly, the ingestible device introduced herein may have an asymmetrically weighted formfactor. As further discussed below, the ingestible device may have a weighted end through which the needle extends in order to address the self-orientation problem. The weighted end may help ensure that the ingestible device properly self-orients itself before medication is administered via the needle.

The weighted end also ensures that the ingestible device is asymmetrically weighted across an axis. As an example, a ballast can be connected to the capsule such that when the ingestible device is in body fluid, the ballast creates a weight differential across a latitudinal axis that vertically bisects the capsule, and the weight differential can cause the ingestible device to naturally position itself against tissue in a target vertical orientation. As another example, a ballast can be connected to the capsule such that when the ingestible device is in body fluid, the ballast creates a weight differential across a longitudinal axis that horizontally bisects the capsule, and the weight differential can cause the ingestible device to naturally position itself against tissue in a target horizontal orientation.

i. Buoyancy Stability

Within the stomach, the ingestible device is representative of a floating structure (also called a “floating body”) whose orientation is changeable yet may need to be in a target orientation in order for medication to be properly administered. A floating body is considered stable if it returns to an equilibrium position after being disturbed. To establish whether a floating body is stable, an analysis of two separate locations must be performed. First, the center of buoyancy must be determined. The center of buoyancy is representative of the centroid of displaced fluid that generates upward force equal to the mass of the displaced fluid. Second, the center of mass must be determined. The center of mass is representative of the centroid of the floating body that generates downward force equal to the mass of the floating body.

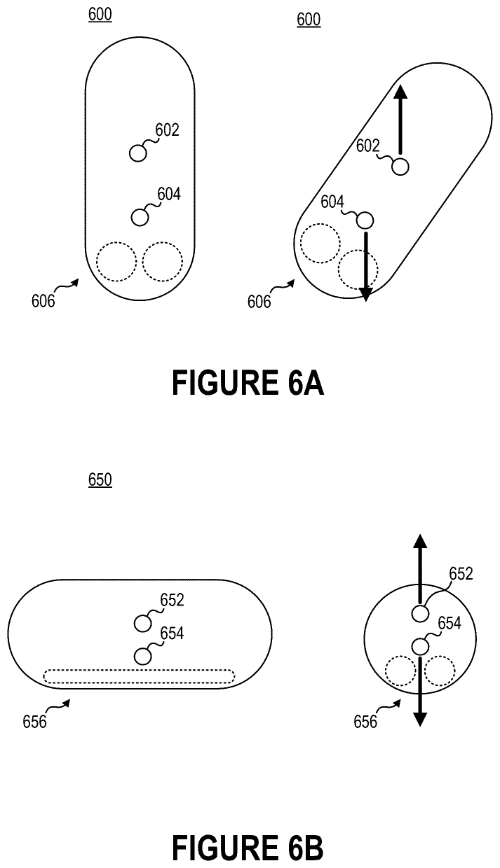

FIG. 6A includes a simplified illustration of the center of buoyancy 602 and center of mass 604 of an ingestible device 600 with a target vertical orientation. FIG. 6B, meanwhile, includes a simplified illustration of the center of buoyancy 652 and center of mass 654 of an ingestible device 650 with a target horizontal orientation. The target vertical orientation may allow for more space along the length of the ingestible device 600 for integration of the needle, while the target horizontal orientation may allow for more resistance to orientation stability independent of the level of body fluid in a target organ cavity. Thus, the target horizontal orientation may be more impervious to variations in the amount of body fluid in the target organ cavity since the ingestible device 650 can be more easily submerged in lower levels of body fluid.

The ingestible devices 600, 650 will be stable so long as the centers of mass 604, 654 are below the centers of buoyancy 602, 652. Said another way, the ingestible devices 600, 650 may be stable if the centers of mass 604, 654 are nearer the weighted end 606, 656 than the centers of buoyancy 602, 652. For a target vertical orientation, an ingestible device 600 with a capsule size of 000 may be stable fluidically if the distance between the center of buoyancy 602 and center of mass 604 is 1.5-3.5 millimeters. For a target horizontal orientation, an ingestible device 650 with a capsule size of 000 may be stable fluidically if the distance between the center of buoyancy 652 and center of mass 654 is 1.0-2.5 millimeters.

ii. Weight Integration

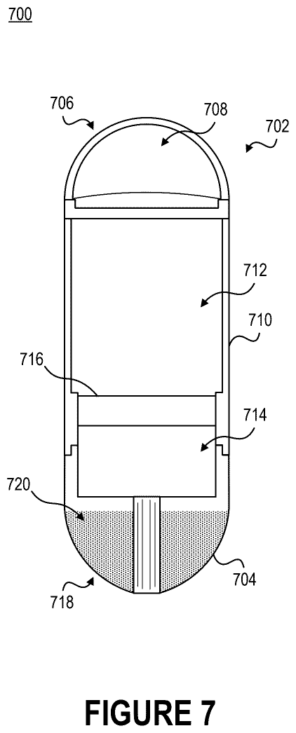

As mentioned above, an important aspect of establishing stability of an ingestible device is having a weighted end. An ingestible device can be ballasted in several ways. FIG. 7 includes an illustration of an ingestible device 700 that includes a capsule 702 to which a ballast 704 is connected. As shown in FIG. 7, the capsule 702 may have (i) a first atraumatically shaped end 706 with a first cavity 708 defined therein and (ii) a cylindrical segment 710 with a second cavity 712 defined therein. The second cavity 712 may be fluidically decoupled from a reservoir 714 in which medication is stored by a seal 716.

When connected to the cylindrical segment 710 of the capsule 702, the ballast 704 may form a second atraumatically shaped end 718. In FIG. 7, the ballast 704 is formed such that its exterior surface has a similar shape as the first atraumatically shaped end 706. Specifically, the ballast 704 has a roughly hemispherical form in FIG. 7. However, the ballast 704 could be another shape. For example, the ballast 704 may be in the form of a hemisphere with a flattened top that is intended to lodge against the surface of the tissue. In some embodiments, the ballast 704 defines at least a portion of the periphery of the reservoir 714. In FIG. 7, for example, the ballast 704 defines the bottom surface of the reservoir 714, as well as parts of the sidewalls of the reservoir 714.

In some embodiments, the ballast 704 is connected to, or incorporated in, the capsule 702 rather than connected to the cylindrical segment 710. For example, the ballast 704 may be weighted components (e.g., spheres or rods) that are arranged within the capsule 702. Examples of such ballasts can be seen in FIGS. 6A-B, where the ballasts are shown using dashed lines. Alternatively, the ballast 704 could be lined or layered along the interior surface of the capsule 702. Assume, for example, that the capsule comprises a cylindrical segment interconnected between atraumatically shaped ends. A high-density material may be lined along the interior surface of one of the atraumatically shaped ends, so as to ballast the entire capsule. Thus, the location of the ballast may dictate or define the weighted end of the ingestible device 700.

The ballast 704 can be fabricated out of various high-density materials. Examples of such materials include metals, metal alloys (e.g., stainless steel, titanium alloys, cobalt-chromium alloys), ceramics (e.g., tungsten carbide), and the like. In some embodiments, the ballast 704 comprises a composite of weighted particles that are molded together. These weighted particles could be molded together with or without a binding agent (also called a “binder”). For example, the ballast 704 may comprise tungsten carbide power that is either dry compressed or molded with an epoxy resin. As another example, the ballast 704 may comprise stainless steel power that is either dry compressed or molded with an epoxy resin. These weighted particles could include metal particles, metal alloy particles, ceramic particles, or any combination thereof. Generally, the ballast 704 comprises a material—or a combination of materials—that has a density of more than 7.0 grams per cubic centimeter (g/cm³). In some embodiments, the ballast 704 comprises a material—or a combination of materials—that has a density of more than 15.0 g/cm³. Tungsten carbide, for example, has a density of roughly 15.63 g/cm³.

As mentioned above, ballasting the ingestible device 700 may result in one atraumatically shaped end being weighted. In FIG. 7, for example, the second atraumatically shaped end 718 is representative of the weighted end. To improve stability, the first atraumatically shaped end 706 may be partially or fully filled with air. Said another way, air may be entrapped within the first cavity 708 of the first atraumatically shaped end 706 to form an “air hat.” Together, the air-filled first cavity 708 and weighted ballast 704 can help the ingestible device 700 more easily establish and then maintain the vertical orientation in the presence of body fluid.

Components are generally housed within the cylindrical segment 710 of the capsule 702. Such components can include a trigger mechanism, plunger mechanism, and springs as shown in FIG. 1. However, the second cavity 712 defined within the cylindrical segment 710 will not be fully occupied. In some embodiments, the second cavity 712 is partially or fully filled with a low-density fluid. Gastric contents have a density close to water (i.e., about 1.004 g/cm³), and the low-density fluid may have a density less than 1.004 g/cm³. Generally, the low-density fluid has a density of less than 1.000 g/cm³. Canola oil, for example, has a density of roughly 0.915 g/cm³. Other examples of suitable low-density fluids include glycerin, some types of gelatin, some medical-grade oils, and combinations thereof.

In some embodiments, air is entrapped in the low-density fluid. As the ingestible device 700 sinks through body fluid due to the ballast 704, the entrapped air will “float” toward the upper end of the second cavity 712. In combination with the ballast 704 and air-filled cavity 708, the entrapped air can assist in orienting the capsule 702 in the vertical orientation without inhibiting sinking. As long as the cumulative density of the ingestible device 700 is greater than the density of the surrounding fluid, the ingestible device 700 will reliably sink. The larger the difference between these densities, the quicker and more reliably the ingestible device 700 will sink.

Note that in some embodiments, the weighted end of the capsule 702 may have a mucoadhesive coating 720 applied thereto. For example, the mucoadhesive coating 720 may be applied along at least part of the exterior surface of the ballast 704. The mucoadhesive coating 720 may comprise any material that is able to improve, at least temporarily, adherence of the ingestible device 700 to the tissue into which medication is to be ejected. In some embodiments, the mucoadhesive coating 720 is also dissolvable. Dissolution of the mucoadhesive material may be helpful in ensuring that the ingestible device 700 readily detaches from the tissue. The mucoadhesive material may be comprised of a material that takes between 20-75 minutes (and preferably 30-50 minutes) to dissolve.

Additionally or alternatively, a shape-memory polymer or shape-memory alloy could be applied to, or integrated within, at least a portion of the weighted side of the capsule 702. For example, a shape-memory polymer could be arranged along the exterior surface of the ballast 704 beneath the mucoadhesive coating 720. Shape-memory polymers are polymeric materials that have the ability to return from a deformed state into the original shape (also called the “natural shape”) when induced by an external stimulus, such as a change in temperature, pH, etc. Accordingly, in embodiments where the mucoadhesive coating 720 dissolves, such dissolution may induce the shape-memory polymer to revert to the original shape as it is exposed to body fluids. Like the mucoadhesive coating 720, the shape-memory polymer can serve as an anchoring feature to further stabilize the ingestible device 700 in its target orientation.

The geometry of a solid dosage form-like the ingestible device 700—can be designed to land the solid dosage form in a certain way along the surface of tissue. Specifically, the geometry can be designed such that the solid dosage form is unstable, and therefore subject to tilting, rocking, or tumbling, in all orientations except one target orientation. However, when the solid dosage form is situated in an organ filled with body fluid, relying entirely on geometry can be problematic. The solid dosage form may be prone to float if the force of buoyancy is greater than the force of gravity, or the solid dosage form may be prone to tilt in unfavorable orientations if the center of buoyancy is near or below the center of mass. By (i) ballasting the ingestible device to create a weighted end and/or (ii) entrapping air within the ingestible device 700, these issues can be addressed, leading to a more stable solid dosage form. By using density to control the center of mass against the center of buoyancy, the ingestible device 700 can keep a more conventional “capsule-like” formfactor. This may be helpful as the conventional formfactor is more user friendly in comparison to oddly shaped solid dosage forms that rely entirely on geometry to establish self-orientation.

iii. Illustrative Example of Asymmetrically Weighted Ingestible Device

To investigate the feasibility of an asymmetrically weighted ingestible device, three different designs were examined. For each design, three weighted components in the form of spheres were adhered within one hemispherical end of a capsule having a spherocylindrical form. These weighted components were comprised of tungsten carbide.

For the first design, no further changes were made. The other hemispherical end and cylindrical segment remained filled with air. For the second design, roughly one-third of the cavity within the cylindrical segment was filled with canola oil while the other hemispherical end remained filled with air. For the third design, nearly the entire cavity within the cylindrical segment was filled with canola oil-leaving enough air for a small bubble several millimeters in diameter—while the other hemispherical end remained filled with air.

Each of the three asymmetrically weighted ingestible devices was placed in fluid having consistent characteristics (e.g., pH of roughly 6.0 and temperature of roughly 72.0° F.) and observed under (i) no agitation, (ii) mild agitation, and (iii) moderate agitation. To achieve mild agitation, the fluid was agitated using a paddle that was driven at 100 revolutions per minute. To achieve moderate agitation, the fluid was agitated using a paddle that was driven at 200 revolutions per minute.

Further details regarding these three designs are provided in Table I.

TABLE I
Estimated values for several different experimental designs of
asymmetrically weighted ingestible device.

| Property | Material | Design 1 | Design 2 | Design 3 |
|---|---|---|---|---|
| Volume (cm³) | Ballast | 0.0953 | 0.0953 | 0.0953 |
| | Air | 1.275 | 0.818 | 0 |
| | Oil | 0 | 0.457 | 1.275 |
| Mass (g) | Ballast | 1.490 | 1.490 | 1.490 |
| | Air | 0.00153 | 0.000985 | 0 |
| | Oil | 0 | 0.418 | 1.166 |
| | Total Capsule | 1.49 | 1.91 | 2.66 |

| | | | |
|---|---|---|---|
| F_{Gravity} (N) | 0.0146 | 0.0187 | 0.0260 |
| F_{Buoyancy} (N), ρ = 1.00 g/cm³ | F_{G} > F_{B} | F_{G} > F_{B} | F_{G} > F_{B} |
| F_{Buoyancy} (N), ρ = 1.09 g/cm³ | F_{G}~F_{B} | F_{G} > F_{B} | F_{G} > F_{B} |

These three designs were examined in several different environments, namely, in air, stationary fluid, mildly agitated fluid, and moderately agitated fluid.

In air, when the first design was positioned in the vertical orientation, the first design was able to remain stably oriented in the vertical orientation. The second design tended to remain in the vertical orientation temporarily and then tip over into the horizontal orientation. Meanwhile, the third design tended to immediately tip over into the horizontal orientation.

In stationary fluid, all three designs allowed for self-orientation. With the first design, the asymmetrically weighted ingestible device was almost entirely submerged beneath the surface of the stationary fluid. With the second and third designs, the asymmetrically weighted ingestible device sunk to the bottom of the vessel.

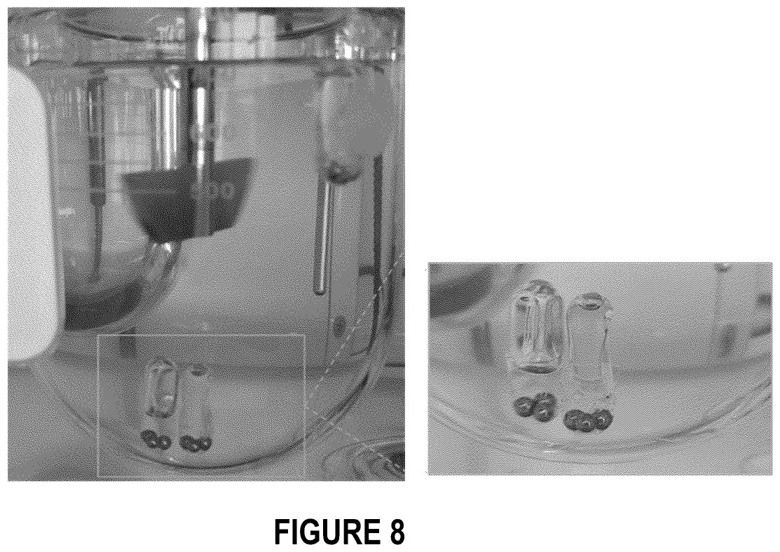

All three designs were able to achieve self-orientation when the fluid was subjected to mild and moderate agitation. With the first design, the asymmetrically weighted ingestible device remained along the top surface of the fluid, circulating along with the fluid flow as dictated by the agitation. With the second design, the asymmetrically weighted ingestible device remained along the bottom of the vessel but circulated along with the fluid flow as dictated by the agitation. Conversely, with the third design, the asymmetrically weighted ingestible device remained “rooted” to the bottom of the vessel even with moderate agitation. FIG. 8 illustrates how the first, second, and third designs reacted in response to agitation of the fluid in the vessel.

Those skilled in the art will recognize that this example is not intended to limit the present disclosure to the embodiments described. Instead, this example has been provided to illustrate several different approaches to achieving self-orientation.

### C. Manufacturing Methodologies

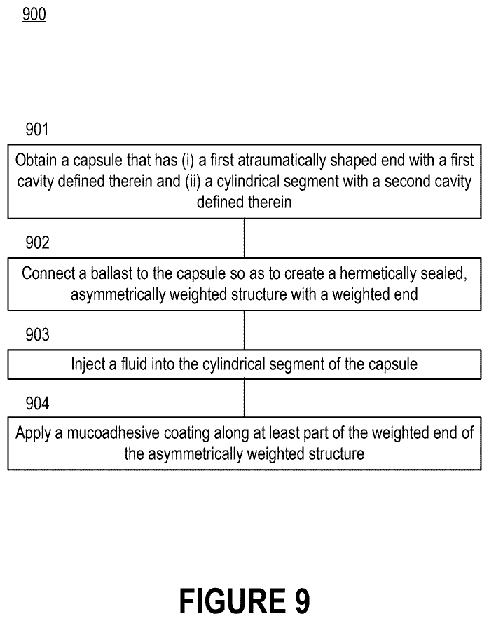

FIG. 9 includes a flow diagram of a process 900 for manufacturing a hermetically sealed, asymmetrically weighted structure that is designed for ingestion by a living body. Initially, a manufacturer can obtain a capsule that has (i) a first atraumatically shaped end with a first cavity defined therein and (ii) a cylindrical segment with a second cavity defined therein (step 901). For example, the manufacturer may produce the first atraumatically shaped end and cylindrical segment and then connect the first atraumatically shaped end to the cylindrical segment. The first atraumatically shaped end and cylindrical segment could be manufactured via injection molding, three-dimensional printing, or another manufacturing process. The first atraumatically shaped end may be connected to the cylindrical segment with an adhesive, or the first atraumatically shaped end may be connected to the cylindrical segment via a soldering process. In some embodiments, these components are designed to complement one another. For example, the first atraumatically shaped end may have a threaded portion that is complementary to a threaded end of the cylindrical segment.

Thereafter, the manufacturer can connect the ballast to the capsule so as to create a hermetically sealed, asymmetrically weighted structure with a weighted end (step 902). As discussed above, the ballast may be connected to the capsule such that the ballast is representative of a second atraumatically shaped end, as shown in FIG. 7. Alternatively, the ballast could be connected to the capsule, for example, along the inner surface of a second atraumatically shaped end. In embodiments where the ballast is positioned entirely inside the capsule, the second atraumatically shaped end may comprise the same material as the first atraumatically shaped end and/or the cylindrical segment. In embodiments where the ballast is positioned entirely inside the capsule, the second atraumatically shaped end may serve as a “shield” or “cover” for the ballast, protecting the ballast from body fluids.

How the ballast is constructed or connected to the capsule may depend on its materials. For example, if the ballast comprises stainless steel, the ballast may be machined into a desired form (e.g., mimicking the lightweight atraumatically shaped end of the capsule). As another example, if the ballast comprises a ceramic such as tungsten carbide, the ballast may be sintered into the desired form. As mentioned above, the ballast may comprise weighted particles that are molded together. These weighted particles could include metal particles, metal alloy particles, ceramic particles, or any combination thereof. These weighted particles could be molded together with a binding agent, or these weighted particles could be compressed together.

As discussed above, the asymmetrically weighted structure is generally formed such that the first atraumatically shaped end is filled partially or entirely with air. The cylindrical segment may be partially or entirely filled with fluid, however. Thus, the manufacturer may inject a fluid into the cylindrical segment (step 903). For example, the manufacturer may insert a needle (e.g., a hypodermic-type needle) into the cylindrical segment, inject the fluid, and then address the hole left by the needle. The hole could be addressed using an adhesive material (e.g., tape or glue), or the hole could be addressed by heating or pressuring the surrounding portion of the cylindrical segment so that material “backfills” the hole. Because gastric contents have a density close to water (i.e., about 1.004 g/cm³), the fluid preferably has a density less than 1.004 g/cm³. Generally, the low-density fluid has a density of less than 1.000 g/cm³.

In some embodiments, air is entrapped in the cylindrical segment of the asymmetrically weighted structure. Following ingestion, the asymmetrically weighted device may sink toward the tissue along the bottom of the stomach due to the ballast, the entrapped air will “float” toward the upper end of the cylindrical segment. In combination with the ballast and air-filled first atraumatically shaped end, the entrapped air can assist in orienting the asymmetrically weighted device in a target orientation. While the air could be injected into the cylindrical segment in a manner similar to the fluid, it is generally easier to inject enough fluid that a known amount of air remains in the cylindrical segment.

Further, the manufacturer may apply a mucoadhesive coating along at least part of the weighted end of the asymmetrically weighted structure (step 904). As discussed above, the mucoadhesive coating may be applied directly to the outer surface of the ballast, or the mucoadhesive coating may be applied to the outer surface of the second atraumatically shaped end if the ballast is contained within the second atraumatically shaped end.

Note that the sequence of steps performed in the process 900 is intended to be illustrative rather than limiting. Unless contrary to physical possibility, the steps may be performed in various sequences and combinations. For example, the fluid could be injected into the cavity in the cylindrical segment before the ballast—or second atraumatically shaped end with ballast connected thereto—is connected to the cylindrical segment. Steps could also be added. For example, coatings (e.g., for inhibiting microbial growth or improving biocompatibility) may be applied to the entire asymmetrically weighted structure prior to packaging and shipping, or coatings may be applied to separate components (e.g., the atraumatically shaped ends, cylindrical segment, ballast, etc.) throughout the process 900. Accordingly, the description of the process 900 is intended to be open ended.

### REMARKS

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling those skilled in the relevant art to understand the claimed subject matter, the various embodiments, and the various modifications that are suited to the particular uses contemplated.

Although the Detailed Description describes certain embodiments and the best mode contemplated, the technology can be practiced in many ways no matter how detailed the Detailed Description appears. Embodiments can vary considerably in their implementation details, while still being encompassed by the specification. Particular terminology used when describing certain features or aspects of various embodiments should not be taken to imply that the terminology is being redefined herein to be restricted to any specific characteristics, features, or aspects of the technology with which that terminology is associated. In general, the terms used in the following claims should not be construed to limit the technology to the specific embodiments disclosed in the specification, unless those terms are explicitly defined herein. Accordingly, the actual scope of the technology encompasses not only the disclosed embodiments, but also all equivalent ways of practicing or implementing the embodiments.

The language used in the specification has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various embodiments is intended to be illustrative, but not limiting, of the scope of the technology as set forth in the following claims.

What is claimed is:

1. A device designed for ingestion by a living body, the device comprising:
a capsule that has
(i) a first atraumatically shaped end with a first cavity defined therein, the first cavity being filled with air, and
(ii) a cylindrical segment with a second cavity defined therein, the second cavity being filled with a fluid with an entrapped air bubble;
a ballast that, when connected to the capsule, forms a second atraumatically shaped end; and
a mucoadhesive coating that is applied along at least part of an exterior surface of the ballast.

2. The device of claim 1, wherein the fluid comprises glycerin, low-viscosity gelatin, a medical-grade oil, or a combination thereof.

3. The device of claim 1, wherein the ballast comprises a metal, metal alloy, or ceramic.

4. The device of claim 1, wherein the ballast comprises a composite of weighted particles that are molded together with an epoxy resin.

5. The device of claim 4, wherein the weighted particles include metal particles, metal alloy particles, ceramic particles, or a combination thereof.

6. The device of claim 1, wherein the ballast has an aperture defined therethrough along a central axis, and wherein, in operation, a needle is actuatable within the aperture along the central axis.

7. An asymmetrically weighted device designed for ingestion by a living body, the asymmetrically weighted device comprising:
a capsule having an axis defined therethrough; and
a ballast that is connected to the capsule such that when the asymmetrically weighted device is in body fluid, the ballast creates a weight differential across the axis that causes the capsule to be naturally positioned against tissue in a targeted orientation.

8. The asymmetrically weighted device of claim 7,
wherein the capsule has (i) a first atraumatically shaped end with air contained therein and (ii) a cylindrical segment through which the axis extends longitudinally, and
wherein when connected to the capsule, the ballast forms a second atraumatically shaped end.

9. The asymmetrically weighted device of claim 8, wherein the targeted orientation is a vertical orientation in which the axis is roughly orthogonal to a surface of the tissue.

10. The asymmetrically weighted device of claim 7,
wherein the capsule has (i) a pair of atraumatically shaped ends and (ii) a cylindrical segment through which the axis extends latitudinally,
wherein the cylindrical segment has an air-filled cavity defined therein along a first side of the axis, and
wherein the ballast is positioned inside the cylindrical segment along a second side of the axis.

11. The asymmetrically weighted device of claim 10, wherein the targeted orientation is a horizontal orientation in which the axis is roughly orthogonal to a surface of the tissue.

12. The asymmetrically weighted device of claim 7, wherein a location of the ballast defines a weighted side of the capsule.

13. The asymmetrically weighted device of claim 12, further comprising:
a mucoadhesive coating that is applied along at least a portion of the weighted side of the capsule.

14. The asymmetrically weighted device of claim 12, further comprising:
a shape memory polymer that is applied to, or integrated within, at least a portion of the weighted side of the capsule.

15. The asymmetrically weighted device of claim 7, wherein at least a portion of the capsule is filled with a fluid that has a density of less than 1 gram per cubic centimeter (g/cm³).

16. The asymmetrically weighted device of claim 7, wherein the ballast comprises a material that has a density of more than 7 grams per cubic centimeter (g/cm³).

17. The asymmetrically weighted device of claim 16, wherein the material has a density of more than 15 grams per cubic centimeter (g/cm³).

18. A method for manufacturing a hermetically sealed structure designed for ingestion by a living body, the method comprising:
obtaining a capsule that has
(i) a first atraumatically shaped end with a first cavity defined therein, the first cavity being filled with air, and
(ii) a cylindrical segment with a second cavity defined therein;
connecting a ballast to the capsule so as to create the hermetically sealed structure, with the ballast being representative of a second atraumatically shaped end,
wherein the ballast comprises a material that has a density of more than then 7 grams per cubic centimeter (g/cm³); and
injecting a fluid having a density of less than 1 grams per cubic centimeter (g/cm³) into the second cavity.

19. The method of claim 18, further comprising:
applying a mucoadhesive coating along at least part of an exterior surface of the ballast.

20. The method of claim 18, wherein said injecting is performed such that an air bubble is entrapped in the fluid.

21. The method of claim 18, wherein said injecting is performed prior to said connecting.
